(19) United States
(12) Patent Application Publication
(10) Pub. No.: US 2026/0145159 A1
(43) Pub. Date: May 28, 2026

(54) MIXED MODE CHARGE INDUCTION ION EXCHANGE (IEX) CHROMATOGRAPHY (MMCIIC) LIGANDS AND METHODS OF USE

(71) Applicant: BIO-RAD LABORATORIES, INC., HERCULES, CA (US)

(72) Inventors: CHRISTOPHER BELISLE, WALNUT CREEK, CA (US); ALEXANDER POLGAR, HERCULES, CA (US); GREGORY SHIMAMURA, HERCULES, CA (US); KANGLING MA, HERCULES, CA (US)

(21) Appl. No.: 19/215,631

(22) Filed: May 22, 2025

Related U.S. Application Data
(60) Provisional application No. 63/651,988, filed on May 25, 2024.

(51) Int. Cl. B01J 20/32; B01D 15/32; B01D 15/38; B01D 15/42; B01J 20/22; B01J 20/289; B01J 20/29; C07D 213/81; C07D 239/42; C07D 241/26; C07K 1/16

(52) U.S. Cl. CPC B01J 20/3255; B01D 15/327; B01D 15/388; B01D 15/424; B01J 20/22; B01J 20/289; B01J 20/29; B01J 20/3206; B01J 20/3219; C07D 213/81; C07D 239/42; C07D 241/26; C07K 1/165

(57) ABSTRACT

The subject invention pertains to mixed mode charge induction IEX chromatography (MMCIIC) ligands and chromatography resins suitable for the purification of proteins from biological sources or biological samples. Methods of making mixed mode charge induction IEX chromatography (MMCIIC) matrices comprising the disclosed ligands and using the disclosed chromatography matrices are also provided.

### CROSS-REFERENCE TO A RELATED APPLICATION

This application claims the benefit of U.S. Provisional Application Ser. No. 63/651,988, filed May 25, 2024, the disclosure of which is hereby incorporated by reference in its entirety, including all figures, tables and amino acid or nucleic acid sequences.

### FIELD OF THE INVENTION

Materials and methods for separating immunoglobulins or other proteins from source liquids, for purposes of purification or isolation, utilizing chromatographic separation techniques are provided. Methods of preparing chromatographic materials suitable for use in such techniques are also provided.

### BACKGROUND

The separation of proteins, such as immunoglobulins or other therapeutic biological agents, from source liquids, such as mammalian bodily fluids or cell culture harvest or supernatants, is of significant commercial interest and value. Also of interest are preparations of proteins in a sufficiently concentrated or purified form for diagnostic, laboratory, and therapeutic uses. However, the purification of proteins often suffers from factors such as low yield, the use of costly separation media (chromatography media), the leaching of separation media (for example, chromatography ligands) into the product, and concerns for the safe disposal of extraneous materials used in the extraction process.

Traditional mixed mode ion exchange chromatography media (MMIEX), such as mixed mode anion exchange (MMAEX) and mixed mode cation exchange (MMCEX) can suffer performance issues due to the static hydrophobic nature of the resins. Step gradients of salts (chaotropes) used in mixed mode (MM) chromatography (i.e. NaCl) often induce the hydrophobic interaction (HIC) mode of binding with the hydrophobic moiety of the MM ligand on the resin. This limits the utility of traditional MMIEX.

The ligands disclosed within this application address at least some of these issues. This disclosure provides mixed mode charge induction IEX chromatography (MMCIIC) ligands based on regio-isomers of aminopyrazine-carboxamido, aminopyrimidine-carboxamido, aminopicolinamido, aminonicotinamido, and aminoisonicotinamido) carboxylic acid structures. In some embodiments, the MMCIIC ligands are based on constitutional isomers of aminopyridine-carboxamido, aminopyrimidine-carboxamido, and aminopyrazine-carboxamido, such as x-aminopyrazine-2-carboxamido (x=3,5,6); x-aminopyrimidine-y-carboxamido (x,y=2,4; 2,5; 4,2; 4,5; 4,6; 5,2; 5,4); x-aminopicolinoyl (x=3,4,5,6); x-aminonicotinoyl (x=2,4,5,6); or x-aminoisonicotinoyl (x=2, 3) carboxylic acids.

The disclosed MMCIIC ligands allow for pH adjustments to modulate 1) the IEX moiety and 2) the hydrophobic nature of the ligands. The nitrogen(s) in the aromatic ring structures become charged at lower pHs and provides charge repulsion (facile elution) of biomolecules that also become positively charged at lower pH. Thus, it permits one to use pH and/or salt to elute proteins from the disclosed chromatography matrices.

### BRIEF SUMMARY

This disclosure provides MMCIIC ligands and chromatography matrices suitable for the purification of proteins from biological sources or samples. Methods of making chromatography matrices and using the disclosed chromatography ligands are also provided.

### BRIEF DESCRIPTION OF THE DRAWINGS

FIG. 1. Elution profile for Nuvia cPrime™ (NcP, Bio-Rad Laboratories, Inc., Hercules, CA). Columns were equilibrated with 20 mM Na₃PO₄ (pH=7; Buffer A). NcP was loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 2. Elution profile for CB491 compared to NcP. Columns were equilibrated with 20 mM Na₃PO₄ (pH=7; Buffer A). NcP and CB491 were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 3. Elution profile for CB490 compared to NcP. Columns were equilibrated with 20 mM Na₃PO₄ (pH=7; Buffer A). NcP and CB490 were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 4. Elution profile for CB482d compared to NcP. Columns were equilibrated with 20 mM Na₃PO+ (pH=7; Buffer A). NcP and CB482d were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 5. Elution profile for CB487 compared to NcP. Columns were equilibrated with 20 mM Na₃PO₄ (pH=7; Buffer A). NcP and CB487 were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 6. Elution profile for CB488 compared to NcP. Columns were equilibrated with 20 mM Na₃PO₄ (pH=7; Buffer A). NcP and CB488 were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 7. Elution profile for CB482j compared to NcP. Columns were equilibrated with 20 mM Na₃PO₄ (pH=7; Buffer A). NcP and CB482j were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 8. Elution profile for CB489 compared to NcP. Columns were equilibrated with 20 mM Na₃PO₄ (pH=7; Buffer A). NcP and CB489 were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 9. Elution profile for CB485i compared to NcP. Columns were equilibrated with 20 mM Na₃PO₄ (pH=7; Buffer A). NcP and CB485i were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 10. Elution profile for CB485j compared to NcP. Columns were equilibrated with 20 mM Na₃PO₄ (pH=7; Buffer A). NcP and CB485j were loaded with a cationic protein standard of myoglobin, ribonuclease A, and cytochrome c. Elution was performed with a salt gradient comprising Buffer A and Buffer B (20 mM Na₃PO₄+1.5 M NaCl (pH=7)).

FIG. 11. Elution profiles for CB489, CB491, CB490, and CB487 compared to NcP using a pH gradient. Two (2) mL columns were equilibrated with 5 column volumes (CV) of Buffer B (50 mM citrate, pH 3.0) followed by 5 column volumes of buffer A (50 mM citrate, pH 6.0). A 250 μL sample of lysozyme (5 mg/ml Lysozyme in Buffer A) was loaded onto the column after equilibration with Buffer A. A pH gradient of 0-100% Buffer B was applied to the column at a flow rate of about 2 mL/minute to elute bound protein (over about 10 CV). The pH gradient was not able to elute the protein on the NcP ligand until the column was stripped with 1M HCl (20 CV). CB489, CB491, CB490, CB482j, and CB487 were stripped with 5 CV of Buffer B.

### DETAILED DISCLOSURE OF THE INVENTION

Unless otherwise stated, the following terms used in this application, including the specification and claims, have the definitions given below. As used in this specification and the appended claims, the singular forms “a”, “an”, and “the” include plural referents unless the content clearly dictates otherwise. Definition of standard chemistry terms can be found in reference works, including Carey and Sundberg (2007) “Advanced Organic Chemistry 5th Ed.” Vols. A and B, Springer Science+Business Media LLC, New York. The practice of the present invention will employ, unless otherwise indicated, conventional methods of synthetic organic chemistry, mass spectrometry, preparative and analytical methods of chromatography, protein chemistry, biochemistry, recombinant DNA techniques and pharmacology.

The terms “biological sample(s)”, “source solution(s)”, or “source liquid(s)” refer to any composition containing a target molecule of biological origin (a “biomolecule”) that is desired to be purified. Non-limiting examples of target molecules include: antibodies, enzymes, growth regulators, clotting factors, transcription factors and phosphoproteins. In some embodiments, the target molecule (biomolecule) to be purified is an antibody or a non-antibody protein. Non-limiting examples of biological samples include serum samples from individuals or cell culture supernatants (e.g., clarified cell culture supernatants). With respect to the purification of biomolecules, such as antibodies, any biological sample that contains the target biomolecule can be used. Non-limiting examples of a source solution or source liquid include unpurified or partially purified antibodies from natural, synthetic, or recombinant sources. Unpurified antibody preparations (source solutions) can come from various sources including, but not limited to, plasma, serum, ascites, milk, plant extracts, bacterial lysates, yeast lysates, or conditioned cell culture media. Partially purified antibody preparations can come from unpurified preparations that have been processed by at least one chromatography, precipitation, other fractionation step, or any combination of the foregoing. In some embodiments, the antibodies have not been purified by protein A affinity prior to purification. Other embodiments utilize antibody preparations that have undergone a preliminary affinity purification step utilizing protein A or protein G.

“Antibody” refers to an immunoglobulin, composite (e.g., fusion protein), or fragmentary form thereof. The term includes but is not limited to polyclonal or monoclonal antibodies of the classes IgA, IgD, IgE, IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” also includes composite forms including but not limited to fusion proteins containing an immunoglobulin moiety. “Antibody” also includes antibody fragments such as Fab, F(ab′)₂, Fv, scFv, Fd, dAb, Fc, whether or not they retain antigen-binding function.

The term “protein” refers to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers (e.g., recombinant proteins).

“Bind-elute mode” refers to an operational approach to chromatography in which the buffer conditions are established so that target molecules and, optionally undesired contaminants, bind to the ligand when the sample is applied to the ligand. Fractionation of the target can be achieved subsequently by changing the conditions such that the target is eluted from the support. In some embodiments, contaminants remain bound following target elution. In some embodiments, contaminants either flow-through or are bound and eluted before elution of the target.

“Flow-through mode” refers to an operational approach to chromatography in which the buffer conditions are established so that the target molecule to be purified flows through the chromatography support comprising the ligand, while at least some sample contaminants are selectively retained, thus achieving their removal from the sample.

The terms “matrix”, “solid support” or “support matrix” can be used interchangeably. In various embodiments, the matrix can be particles, a membrane or a monolith, and by “monolith” is meant a single block, pellet, or slab of material. Particles, when used as a matrix (or “matrices”), can be spheres or beads, either smooth-surfaced or with a rough or textured surface and can, optionally, contain pores (i.e., the particles may contain pores or the particles contain no pores). Many, and in some cases all, of the pores are through-pores, extending through the particles to serve as channels large enough to permit hydrodynamic flow or fast diffusion through the pores. When in the form of spheres or beads, the median particle diameter, where the term “diameter” refers to the longest exterior dimension of the particle, is preferably within the range of about 25 microns to about 150 microns. The spheres or beads can have pores of a median diameter of 0.5 micron or greater, optionally with substantially no pores of less than 0.1 micron in diameter. In certain embodiments of the invention, the median pore diameter ranges from about 0.5 micron to about 2.0 microns. The pore volume can vary, although in many embodiments, the pore volume will range from about 0.5 to about 2.0 cc/g. Disclosures of matrices meeting the descriptions in this paragraph and the processes by which they are made are found in Hjerten et al., U.S. Pat. No. 5,645,717, Liao et al., U.S. Pat. No. 5,647,979, Liao et al., U.S. Pat. No. 5,935,429, and Liao et al., U.S. Pat. No. 6,423,666. Examples of monomers that can be polymerized to achieve useful matrices are vinyl acetate, vinyl propylamine, acrylic acid, methacrylate, butyl acrylate, acrylamide, methacrylamide, vinyl pyrrolidone (vinyl pyrrolidinone), with functional groups in some cases. Crosslinking agents are also of use in many embodiments, and when present will generally constitute a mole ratio of from about 0.1 to about 0.7 relative to total monomer. Examples of crosslinking agents are dihydroxyethylenebisacrylamide, diallyltartardiamide, triallyl citric triamide, ethylene diacrylate, bisacrylylcystamine, N,N′-methylenebisacrylamide, and piperazine diacrylamide.

The chromatography ligands are linked to the matrix via a linker to form a “chromatography resin”, “chromatography medium” or “chromatography matrix” (terms that can be used interchangeably). Linkage of the chromatography ligand to the matrix will depend on the specific matrix used and the chemical group to be linked to the matrix. Ligands can be linked to the matrix by performing a reaction between a functional group on the ligand, for example an amine group, and a functional group on the matrix, for example, an aldehyde or diol group. For matrices that do not have a suitable functional group, the matrix is reacted with a suitable activating reagent to create a suitable functional group to which the chromatography ligand can be attached.

For purposes of the formation of a linkage with the chromatography ligand, the inclusion of monomers with vicinal diols attached to the matrix is useful. One monomer example is allyloxy propanediol (3-allyloxy-1,2-propanediol). Vicinal diol monomers can be used with other monomers to prepare copolymers. The diol group density in the polymers produced from diol-containing monomers can vary widely, such as for example densities within a range of from about 100 to 1,000 μmol/mL (i.e., micromoles of diol per milliliter of packed beads), and in many cases a range of from about 200 to 300 μmol/mL. An example of a matrix that meets this description are derivatives of UNOsphere Diol, such as UNOsphere Aldehyde—see General Reaction Scheme. To couple a pendant amine-containing ligand to a matrix with exposed vicinal diols, the diols can be oxidized to aldehyde groups, and the aldehyde groups can then be coupled to amine groups to form secondary amino linkages, all by conventional chemistry techniques well known in the art. In some embodiments, the matrix comprises a diol, which is converted to an aldehyde, e.g., by conversion with NaIO4. The primary amine of the ligand can be linked to an aldehyde on the matrix by a reductive amination reaction by the scheme provided in Example 1.

As used herein, the term “linker” refers to a molecule having 1-10 carbon atoms, preferably an alkyl group. The linker has a neutral charge and can include cyclic groups. The linker links the chromatographic ligand to the chromatography matrix. As used herein, the term “alkyl” refers to a straight or branched, saturated, aliphatic radical having between 1-10 carbon atoms. For example, C₁-C₆ alkyl includes, but is not limited to, methyl, ethyl, propyl, isopropyl, butyl, isobutyl, sec-butyl, tert-butyl, pentyl, isopentyl, and/or hexyl. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two chemical groups together.

This disclosure provides a variety of chromatography ligands, including various examples described in Table 1. Table 1 provides non-limiting exemplary ligand structures as well as an exemplary ligand-matrix structures. As would be apparent to those skilled in the art, the linker (discussed above) attaching the ligand to the solid support (matrix) can be an alkyl group between 1 and 10 carbons in length, preferably between 1 and 5 carbons in length, or 1 to 3 carbons in length and is represented by a line between the support and the amine group on the ligand as illustrated herein. With respect to this disclosure, ligands are attached to a black bead and line represents the solid support and the linker to which the ligand is attached (e.g., see Table 1 and the disclosure, below).

As used herein, the term “about” or “approximately” means within an acceptable error range for the particular value as determined by one of ordinary skill in the art, which will depend in part on how the value is measured or determined, i.e., the limitations of the measurement system. In the context of reagent and/or analyte concentrations, the term “about” or “approximately” can mean a range of around a given value of 0-20%, 0 to 10%, 0 to 5%, or 0-1% of a given value (e.g., ±20%, ±10%, ±5% or ±1% of a given value). In the context of pH measurements, the terms “about” or “approximately” permit a variation of ±0.1 or ±0.2 unit from a stated value.

The disclosed ligands use in MMCIIC have the following general structures:

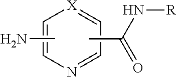

where X is CH or N; or

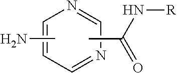

and for each ligand

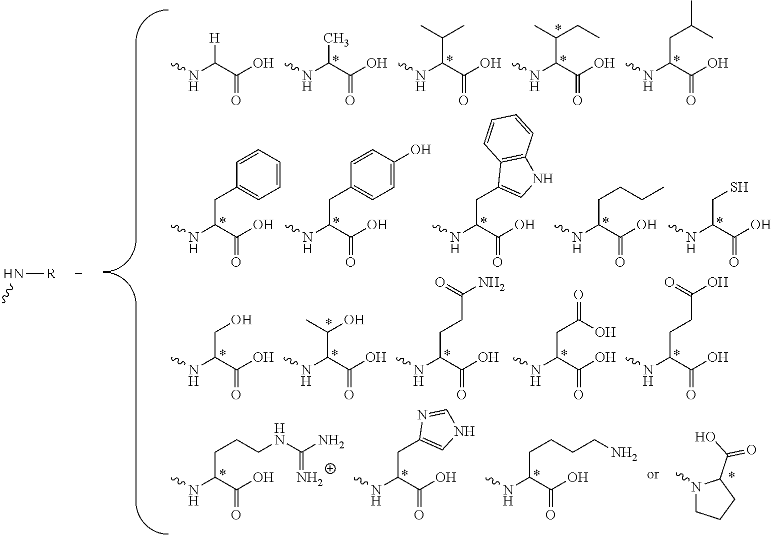

and the * represents a stereogenic carbon atom.

The disclosed MMCIIC chromatography matrices have the following general structures:

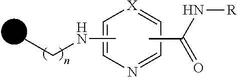

where n is 1, 2, 3, 4, 5, 6, 7, 8, 9, or 10, X is CH or N; or

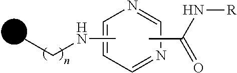

where n is 1, 2, 3, 4, 5, 6, 7, 8, 9, or 10; or

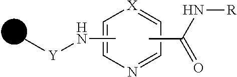

where Y represents an alkyl linker that is a straight or branched, saturated, aliphatic divalent radical having between 1-10 carbon atoms, X is CH or N; or

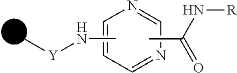

where Y represents an alkyl linker that is a straight or branched, saturated, aliphatic divalent radical having between 1-10 carbon atoms; and for each ligand attached to the solid support:

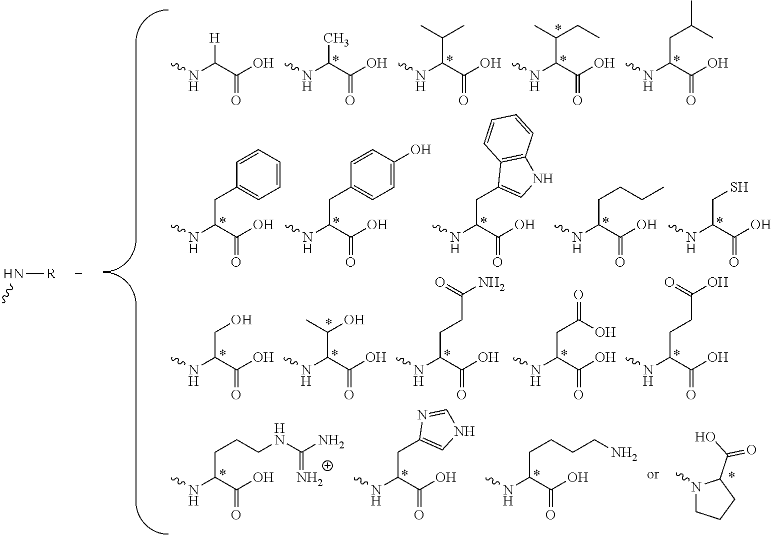

and the * representing a stereogenic carbon atom.

The disclosed ligands can be synthesized by standard chemical reactions. In some embodiments, amino acids (pure D-amino acid, pure L-amino acid, or a racemic mixture of an amino acid) can be reacted with 3-aminopyrazine-2-carboxylic acid, 5-aminopyrazine-2-carboxylic acid, 6-aminopyrazine-2-carboxylic acid, the various regio-isomers of 1) aminopyrimidine-carboxylic acids, 2) aminopicolinic acids, 3) aminonicotinic acid or 4) aminoisonicotinic acid to form a ligand in accordance with this disclosure. In some embodiments, amino acids (pure D-amino acid, pure L-amino acid, or a racemic mixture of an amino acid) can be reacted with constitutional isomers of aminopyridine-carboxylic acid, aminopyrimidine-carboxylic acid, and aminopyrazine-carboxylic acid, such as x-aminopyrazine-2-carboxylic acid (x=3, 5, 6); x-aminopyrimidine-y-carboxylic acid (x,y=2,4; 2,5; 4,2; 4,5; 4,6; 5,2; 5,4); x-aminopicolinic acid (x=3, 4, 5, 6); x-aminonicotinic acid (x=2, 4, 5, 6); or x-aminoisonicotinic acid (x=2, 3) to form a ligand in accordance with this disclosure. These ligands can then be immobilized on a solid support to form a chromatography resin. As would be apparent, the chromatography resin can, in some cases, be a chiral resin.

Protein purification utilizing a chromatography resin in accordance with the present invention can be achieved by conventional means known to those of skill in the art. Examples of proteins include but are not limited to antibodies, enzymes, growth regulators, clotting factors, transcription factors and phosphoproteins. In many such conventional procedures, the chromatography resin prior to use is equilibrated with a buffer at the pH that will be used for the binding of the target protein (e.g., an antibody or a non-antibody protein). Equilibration can be done with respect to all features that will affect the binding environment, including ionic strength and conductivity when appropriate.

In some embodiments, the chromatography resins described herein can be used in “bind-elute” mode to purify a target protein from a biological sample. In some embodiments, following binding of the target protein to the chromatography resin, a change in pH and/or salt can be used to elute the target protein.

In some embodiments, once the chromatography resin is equilibrated, a sample containing the target protein (e.g., a biological sample) is loaded onto the chromatography resin. The sample is maintained at a pH of between about 4.5 and about 8 with an appropriate buffer, allowing the target protein to bind to the chromatography resin. Notably, it has been found that the mixed mode chromatography resins described herein function with solutions having salt concentrations in the range of salt concentrations of cell cultures (e.g., 50-300 mM, or about 100-150 mM). Thus, in some embodiments, the protein is loaded to the chromatography resin under such salt concentrations.

In some embodiments, the chromatography resin is then washed with a wash buffer to remove contaminating proteins or other molecules that may have been present in the source liquid, while retaining the target molecule. The wash buffer may be at the same or different pH or salt concentration as the source material. In some embodiments, the wash buffer may contain salts or other solutes that may not exist in the source material.

The bound target protein (e.g., antibody or non-antibody protein, as desired) can be subsequently eluted. In some embodiments, the protein is then eluted with an elution buffer at a pH above about 4.5, about 5.0, about 6.0, or about 7.0. Illustrative pH ranges, as cited above, are a pH of about 4.5 to about 8 for the binding and washing steps, and pH of about 4.5 to about 8, about 5.0 to about 8.0, about 6.0 to about 8.0, or about 7.0 to about 8.0 for the elution step. In certain embodiments, the binding and washing steps are performed with the inclusion of a salt in the sample and wash liquids. Examples of salts that can be used for this purpose are alkali metal and alkaline earth metal halides, notably sodium and potassium halides, and as a specific example sodium chloride. The concentration of the salt can vary; in most cases, an appropriate concentration will be one within the range of about 10 mM to about 1.5M. As will be seen in the working examples below, optimal elution conditions for some proteins will involve a buffer with a higher salt concentration than that of the binding buffer, and in other cases by a buffer with a lower salt concentration than that of the binding buffer. Elution can be performed as a gradient where buffer conditions transition continuously between a non-eluting buffer and the elution buffer or as a step where buffers are switched abruptly. The optimal choice in any particular case is readily determined by routine experimentation.

In some embodiments, the chromatography resins described herein can be used in “flow-through” mode to purify a target protein from a biological sample. In such instances, once the chromatography resin is equilibrated, a sample containing the target protein (e.g., a biological sample) is loaded onto the chromatography resin. The conditions (e.g., pH and ionic strength) of the equilibration buffer and the loaded sample are controlled such that the target protein flows through the resin or is otherwise unretained while contaminating molecules are retained by binding with the resin. A wash step may follow the sample loading step to flush out molecules of the target protein that are unbound but remaining within the voids of the resin bed or weakly bound to the resin. The contaminants that are bound to the resin may be stripped from it and collected separately from the target molecule or disposed of in order to regenerate the resin for use in additional cycles.

The chromatography resin can be utilized in any conventional configuration, including packed columns and fluidized or expanded-bed columns, and by any conventional method, including batchwise modes for loading, washes, and elution, as well as continuous or flow-through modes. The use of a packed flow-through column is particularly convenient, both for preparative-scale extractions and analytical-scale extractions. A column may, thus, range in diameter from 0.5 cm to 1 m, and in height from 1 cm to 30 cm or more. In some embodiments, a flow-through column can contain a mixture of particles, each particle comprising one of the chromatography ligands disclosed herein. In other embodiments, one or more chromatography ligands can be immobilized on a solid support, such as a particle, membrane or monolith to provide a chromatography resin that provides a mixture of chromatography ligands disposed on the solid support.

Additional claimable subject matter:
1. A ligand of the structure:

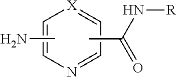

where X is CH or N; or

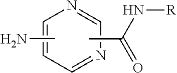

and for each ligand:

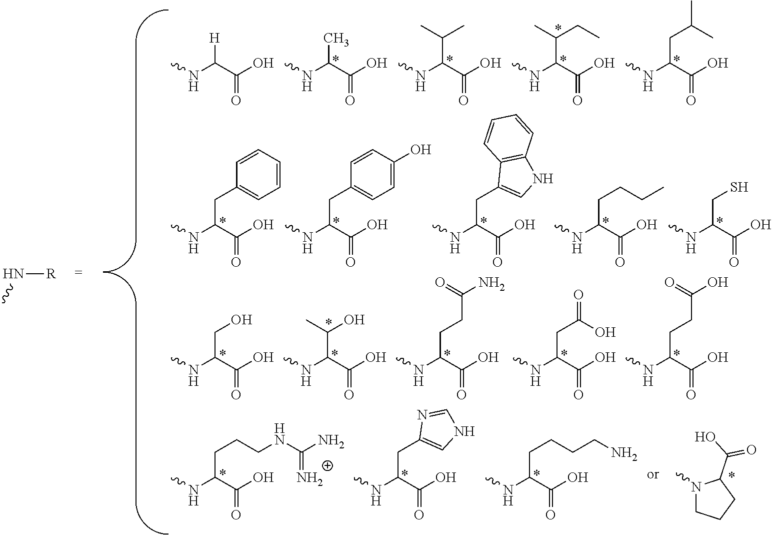

and the * represents a stereogenic carbon atom.
2. The ligand according to embodiment 1, wherein said ligand is:

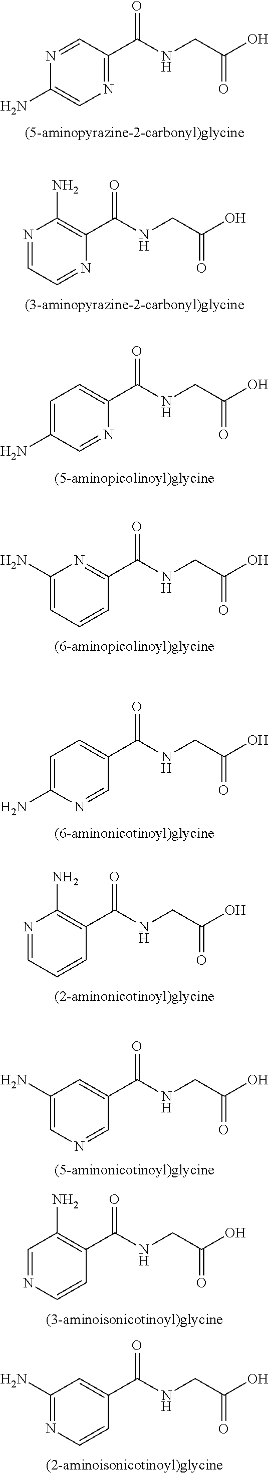

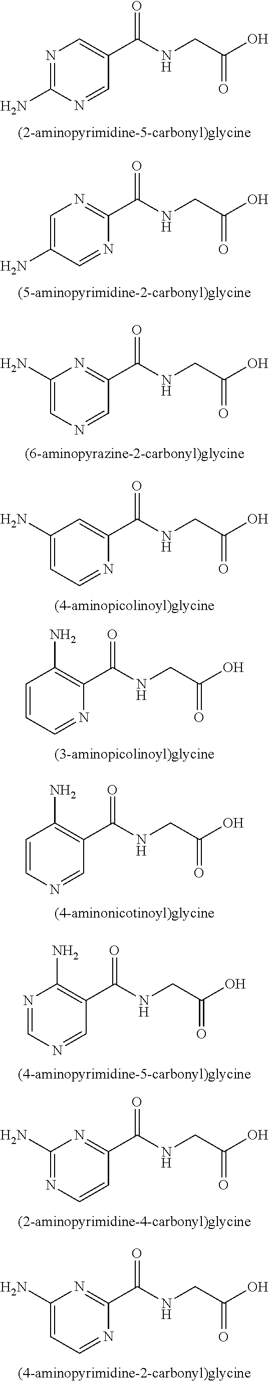

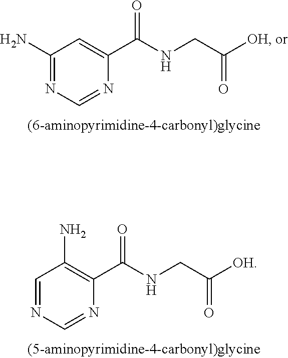

3. The ligand according to embodiment 1, wherein the ligand is a chiral ligand and the stereogenic carbon of the group R that is alpha to the amide group has a D-configuration or has an L-configuration.
4. A mixed mode charge induction IEX chromatography (MMCIIC) chromatography resin comprising:
a support matrix, an optional linker and one or more ligand according any one of embodiments 1-3, the one or more ligand being coupled to the support matrix via the primary amine group of the ligand to form a mixed mode charge induction IEX chromatography (MMCIIC) resin.
5. The MMCIIC chromatography resin according to embodiment 4, wherein the one or more ligand is a chiral ligand and the stereogenic carbon of the group R that is alpha to the amide group has a D-configuration or has an L-configuration.
6. The MMCIIC chromatography resin according to embodiment 4 or embodiment 5, said one or more ligand being attached to the support matrix by a linker.
7. The MMCIIC chromatography resin according to embodiment 6, said one or more ligand being attached to the support matrix by an alkyl linker that is a straight or branched, saturated, aliphatic divalent radical having between 1-10 carbon atoms.
8. The MMCIIC chromatography resin according to embodiment 6, wherein the alkyl linker is a methyl, ethyl, propyl, isopropyl, butyl, isobutyl, sec-butyl, tert-butyl, pentyl, isopentyl, or hexyl divalent radical.
9. A mixed mode charge induction IEX chromatography (MMCIIC) chromatography medium comprising a solid support and a ligand and having the formula:

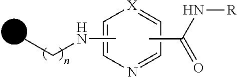

where n represents an alkyl group of 1 to 10 carbon atoms covalently bonded to the solid support (sphere) and the nitrogen atom, X is CH or N; or

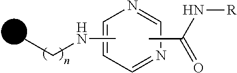

where n represents an alkyl group of 1 to 10 carbon atoms covalently bonded to the solid support (sphere) and the nitrogen atom; or

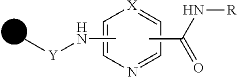

where Y represents an alkyl linker that is a straight or branched, saturated, aliphatic divalent radical having between 1-10 carbon atoms that is covalently bonded to the solid support (sphere) and nitrogen atom, X is CH or N; or

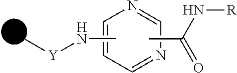

where Y represents an alkyl linker that is a straight or branched, saturated, aliphatic divalent radical having between 1-10 carbon atoms that is covalently bonded to the solid support (sphere) and nitrogen atom; and

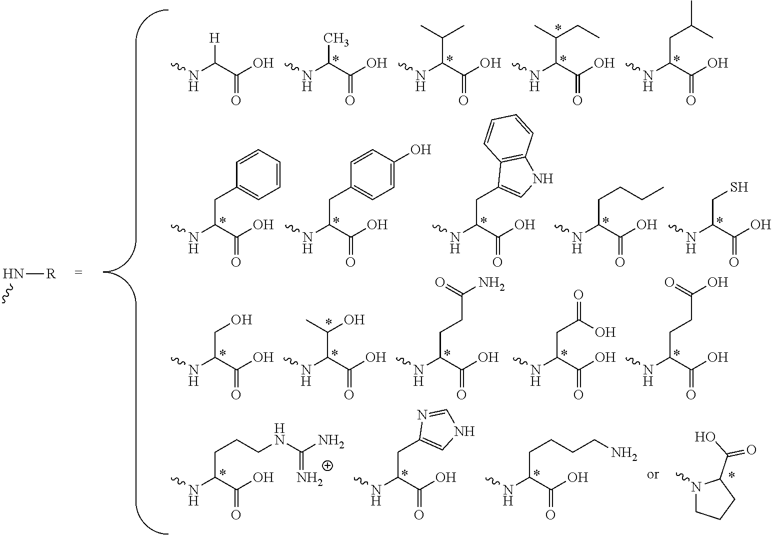

and the * representing a stereogenic carbon atom.
10. The mixed-mode chromatography medium according to embodiment 9, wherein the ligand is a chiral ligand and the stereogenic carbon of the group R that is alpha to the amide group has a D-configuration or has an L-configuration.
11. The mixed-mode chromatography medium according to embodiment 9 or embodiment 10, wherein the mixed mode chromatography medium is selected from the group consisting of:

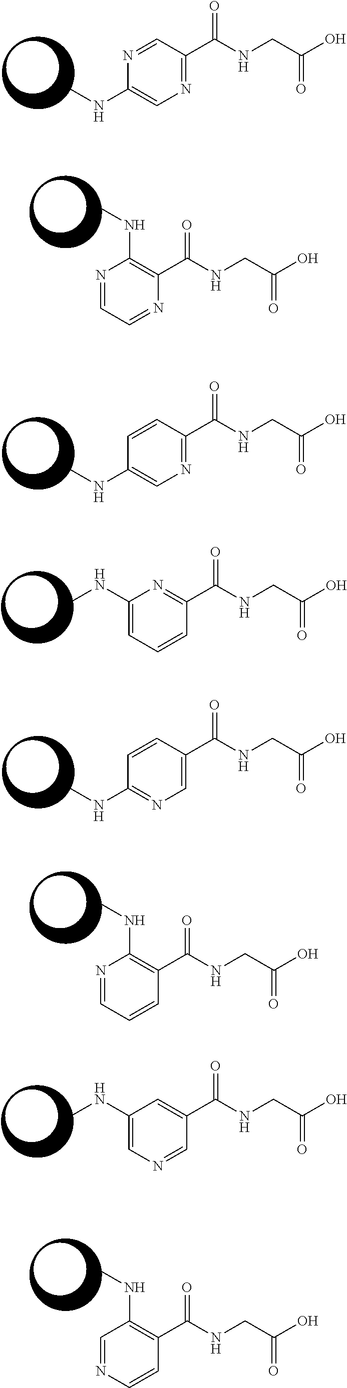

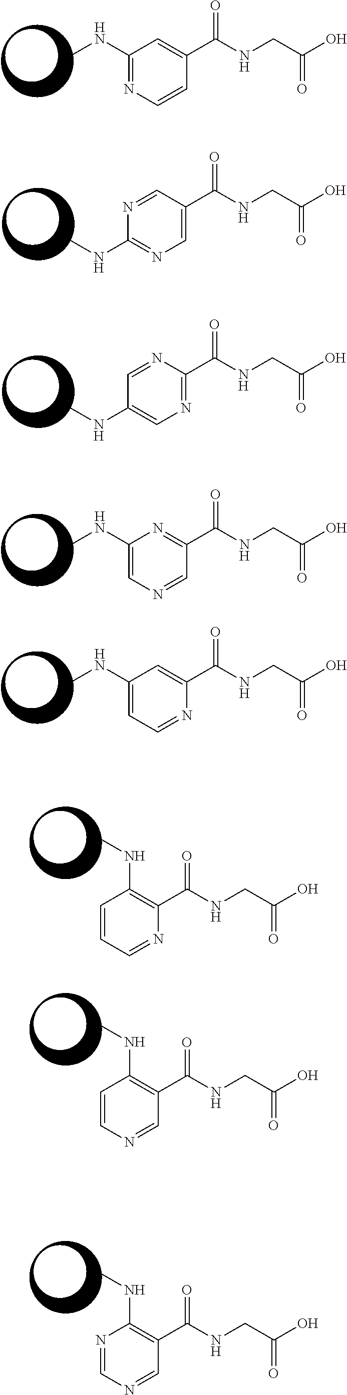

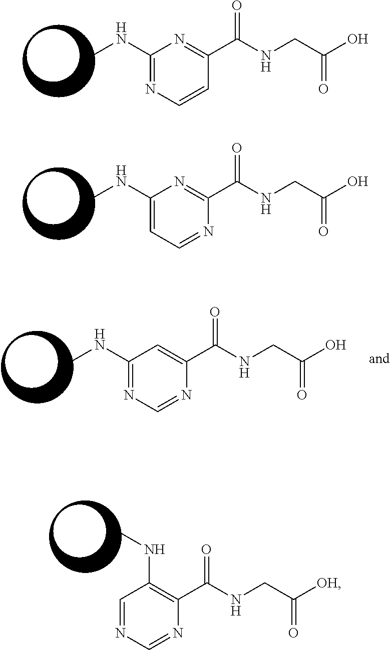

wherein the black bead and line represents the solid support and linker covalently bonded to the nitrogen atom.
12. A method for manufacturing a mixed-mode chromatography medium, said method comprising: (a) oxidizing diol groups on diol-functionalized solid support, thereby converting said diol-functionalized solid support to an aldehyde-functionalized solid support; and (b) coupling one or more amine-functionalized ligand according to any one of embodiments 1-3 to said aldehyde-functionalized solid support.
13. The method according to embodiment 12, wherein a single amine-functionalized ligand is coupled to said aldehyde-functionalized solid support.
14. The method according to embodiment 12 or 13, wherein the ligand(s) is/are a chiral ligand and the stereogenic carbon of the group R that is alpha to the amide group has a D-configuration or has an L-configuration.
15. A method for purifying a protein from a source solution, said method comprising: (a) contacting said source solution with a mixed mode charge induction IEX chromatography (MMCIIC) mixed-mode chromatography medium according to any one of embodiments 9-11 or a chromatography resin according to any one of embodiments 4-8 under conditions that permit said protein to bind to said MMCIIC medium or chromatography resin; and (b) eluting said protein so bound from said MMCIIC medium or chromatography resin.
16. The method according to embodiment 15, wherein the protein is eluted using a pH gradient.
17. The method according to embodiment 15, wherein the protein is eluted using a salt gradient.
18. A method for purifying a protein from a source solution, said method comprising: (a) contacting said source solution with a mixed mode charge induction IEX chromatography (MMCIIC) mixed-mode chromatography medium according to any one of embodiments 9-11 or a chromatography resin according to any one of embodiments 4-8 under conditions which cause the protein to not bind to said MMCIIC medium or chromatography resin; and (b) collecting said protein without it having been bound to said solid support, separating it from molecules that are bound.
19. The method according to any one of embodiments 15-18, wherein the chromatography resin or medium comprises a ligand or ligands that are chiral and the stereogenic carbon of the group R that is alpha to the amide group has a D-configuration or has an L-configuration.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Coupling of Ligands to a Solid Support

### General Reaction Scheme

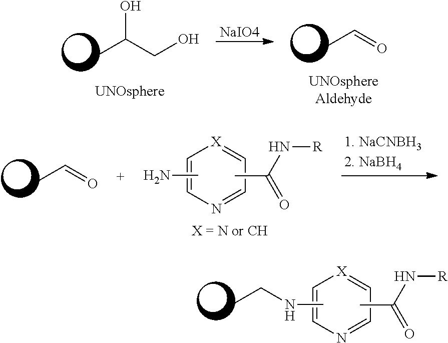

The general reaction scheme, illustrated above, utilizes UNOsphere aldehyde, derived from UNOsphere diol through oxidation with sodium periodate, and ligand structure where HN—R corresponds to various functionalities disclosed herein, for example, any HN—R group disclosed herein.

### General Procedure for Resin CB487, CB488, CB489, CB490, and CB491:

The ligand (˜1.3-2.3 mmol eq.) was dissolved in 10 mL 50% isopropyl alcohol/water (v/v), adjusting the pH of the solution to 1.9-2.1. An equal volume (10 mL) of UNOsphere aldehyde was subsequently added to the solution and vigorously stirred at 25° C. for 30 minutes. This was followed by the addition of 0.2 g sodium cyanoborohydride, and the mixture was stirred vigorously at 25° C. for overnight. Subsequently, 0.1 g sodium borohydride was added and stirred at 25° C. for 1 hour. The UNOsphere-ligand chromatography matrix was then washed with 3 column volumes of 50% isopropyl alcohol, followed by 3 column volumes of 0.1N NaOH, and finally with 15 column volumes of water. The ionic capacity of each resin was in the range of 30-70 μmol/mL. The structure and ionic capacity of resin CB487, CB488, CB489, CB490, and CB491 are listed in Table 1.

### General Procedure for Resin CB485i and CB485j:

The ligand (˜1.3-2.3 mmol eq.) was dissolved in 10 mL 50% dimethyl sulfoxide/water (v/v), adjusting the pH of the solution to 1.9-2.1. An equal volume (10 mL) of UNOsphere aldehyde was subsequently added to the solution and vigorously stirred at 25° C. for 30 minutes. This was followed by the addition of 0.2 g sodium cyanoborohydride, and the mixture was stirred vigorously at 25° C. for overnight. Subsequently, 0.1 g sodium borohydride was added and stirred at 25° C. for 1 hour. The UNOsphere-ligand chromatography matrix was then washed with 3 column volumes of 50% dimethyl sulfoxide, followed by 3 column volumes of 0.1N NaOH, and finally with 15 column volumes of water. The structure and ionic capacity of resin CB485i and CB485j are listed in Table 1.

### General Procedure for Resin CB482c and CB482e:

The ligand (˜1.3-2.3 mmol eq.) was dissolved in 10 ml of water. An equal volume (10 mL) of UNOsphere aldehyde was subsequently added to the solution and vigorously stirred at 25° C. for 30 minutes. This was followed by the addition of 0.2 g sodium cyanoborohydride, and the mixture was stirred vigorously at 25° C. for overnight. Subsequently, 0.1 g sodium borohydride was added and stirred at 25° C. for 1 hour. The UNOsphere-ligand chromatography matrix was then washed with 3 column volumes of 0.1N NaOH, and 15 column volumes of water. The structure and ionic capacity of resin CB482c and CB482e are listed in Table 1.

### Example 2—General Procedure for Protein Separation

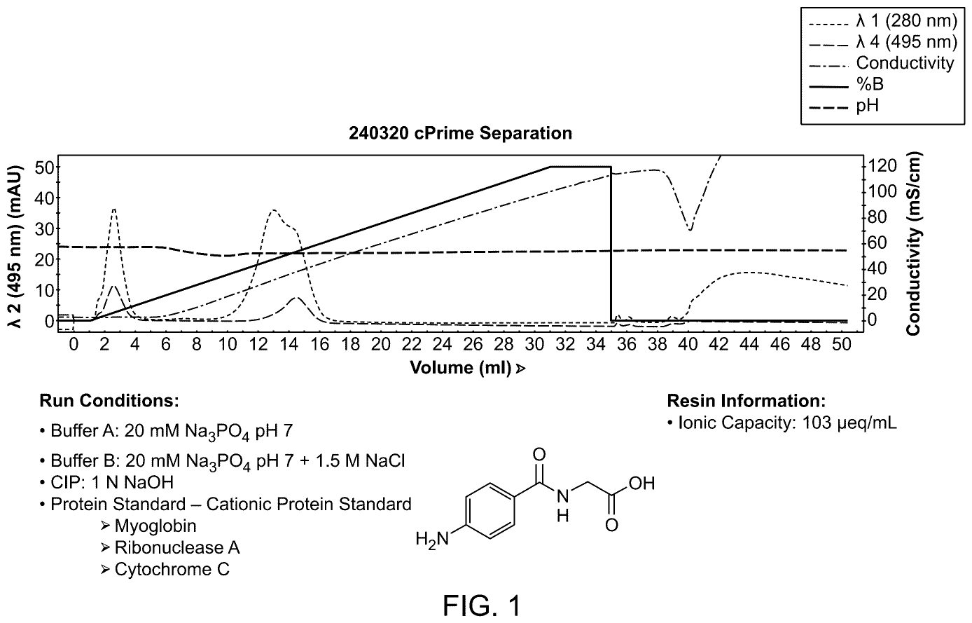

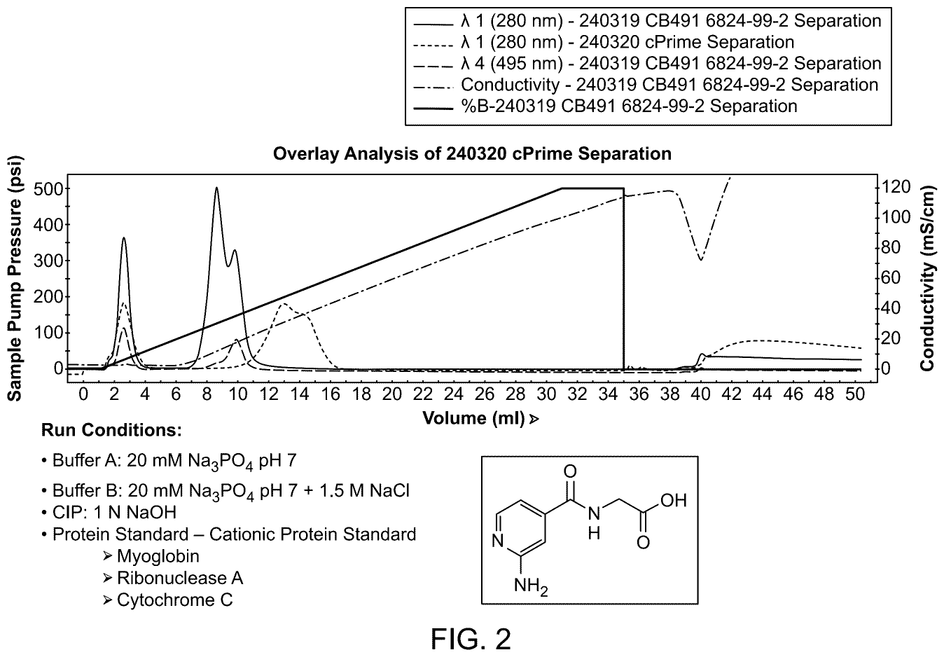

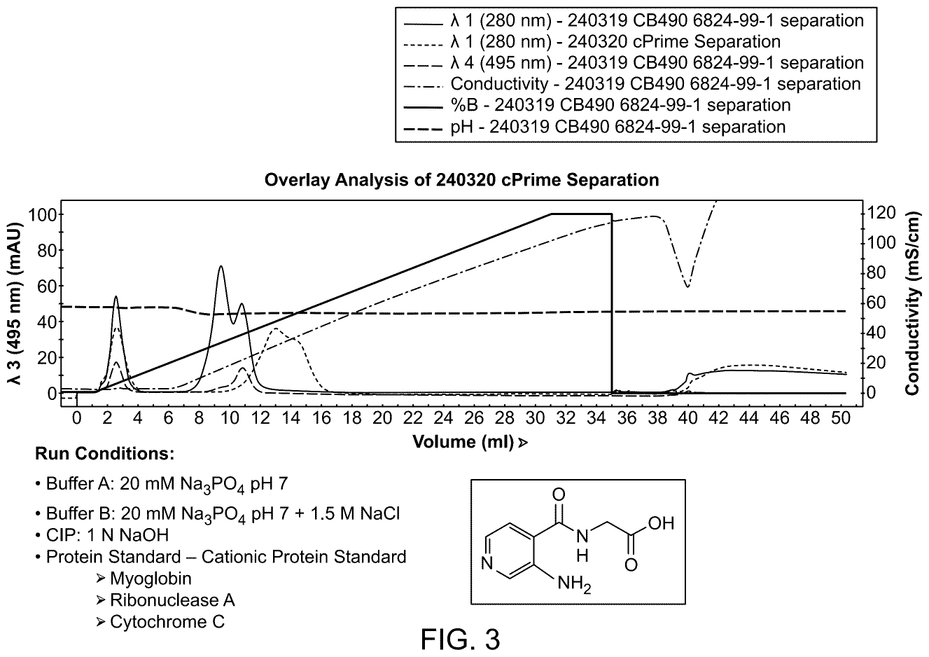

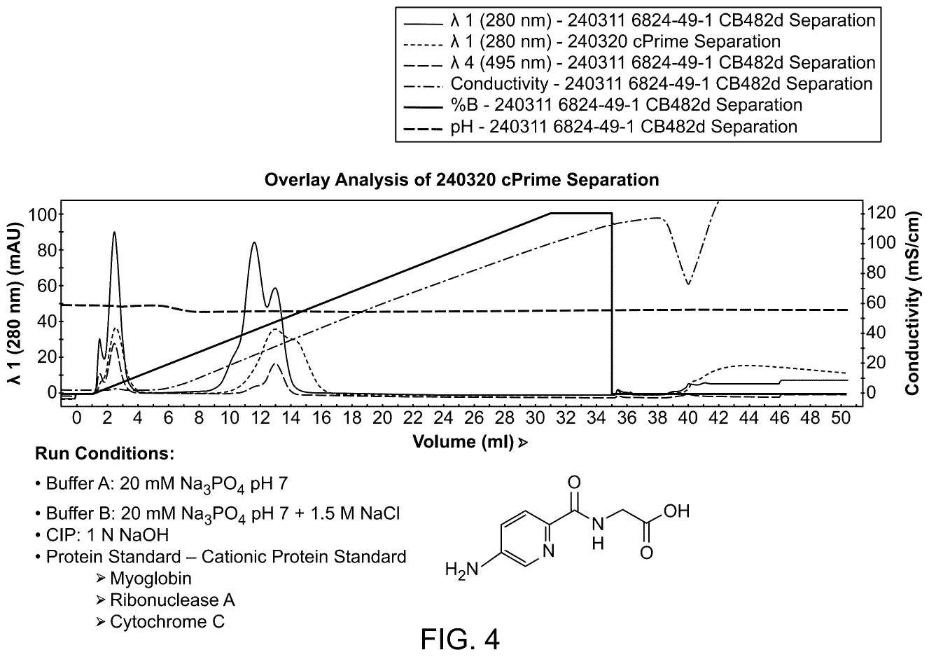

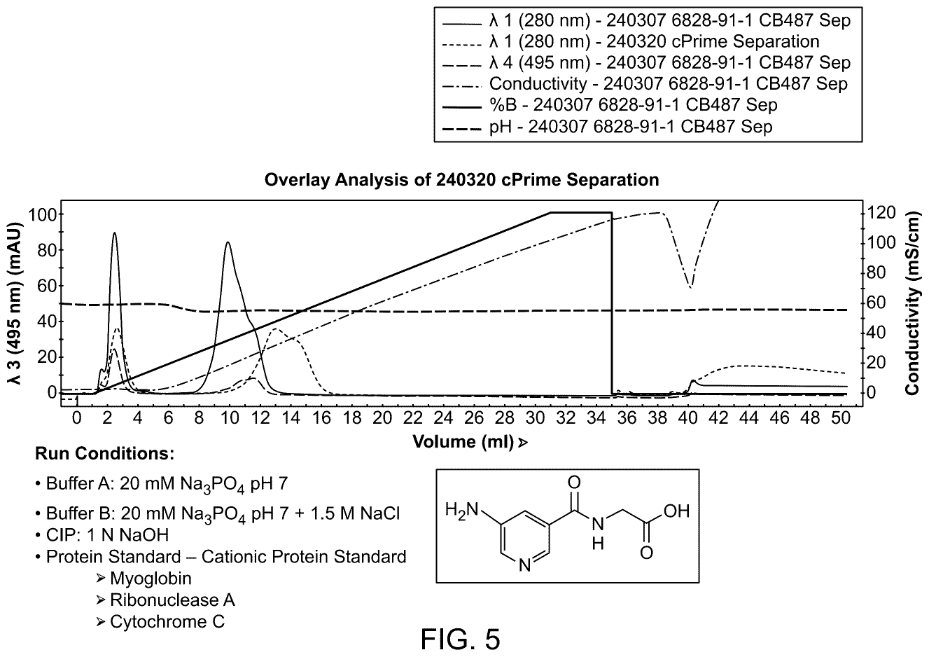

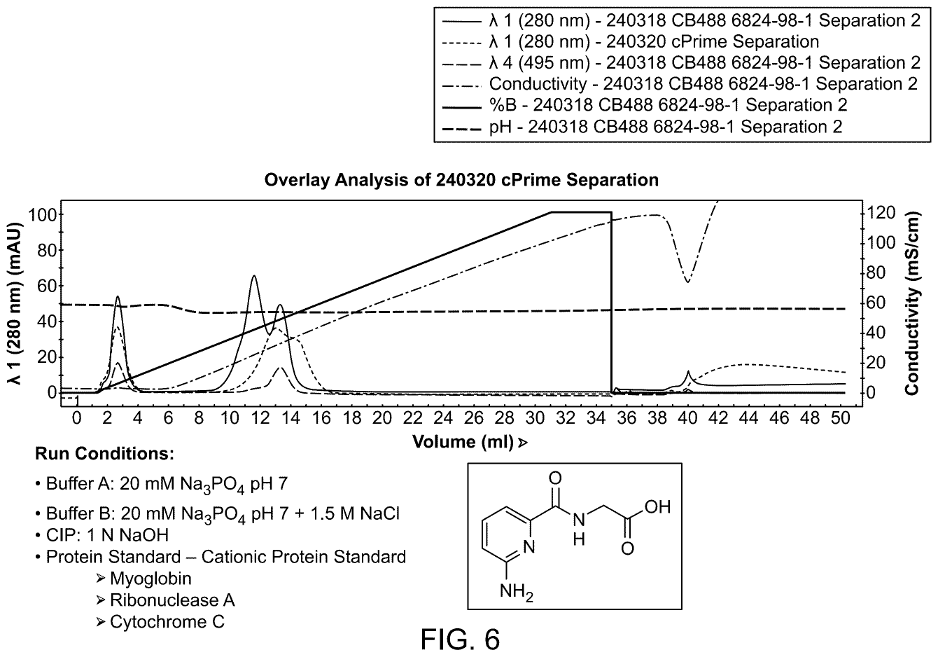

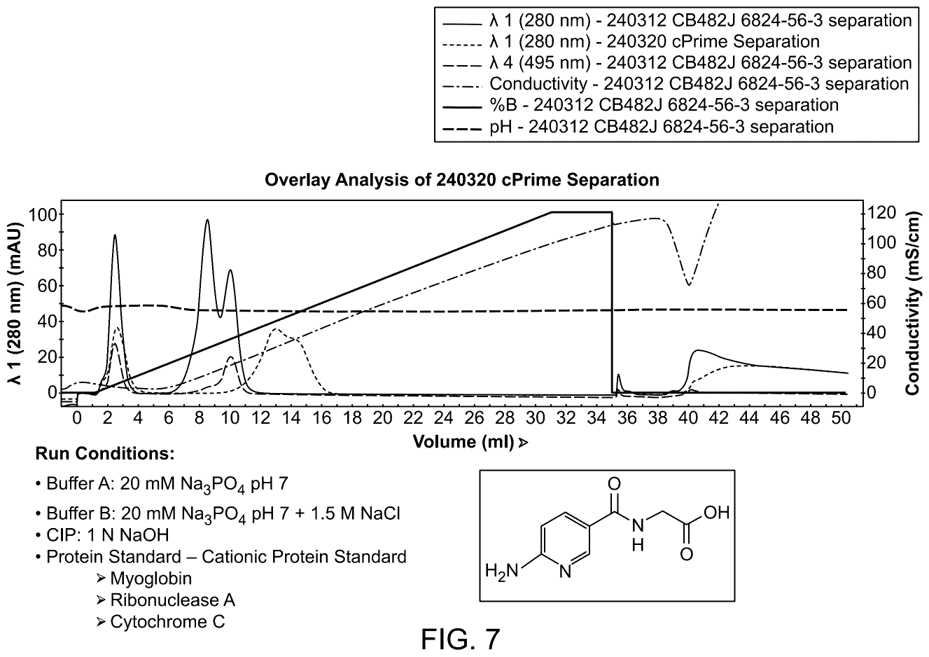

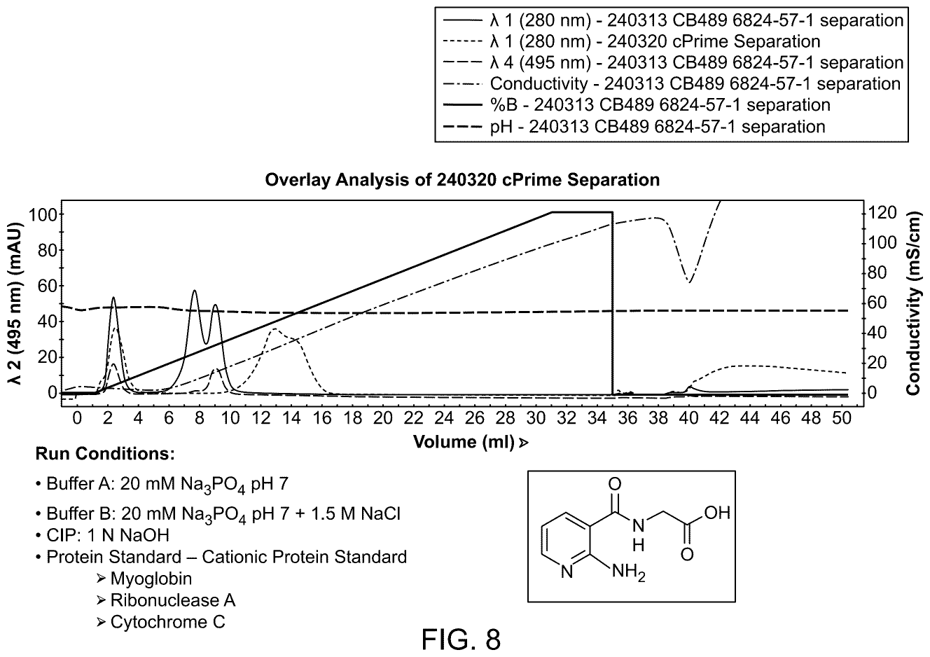

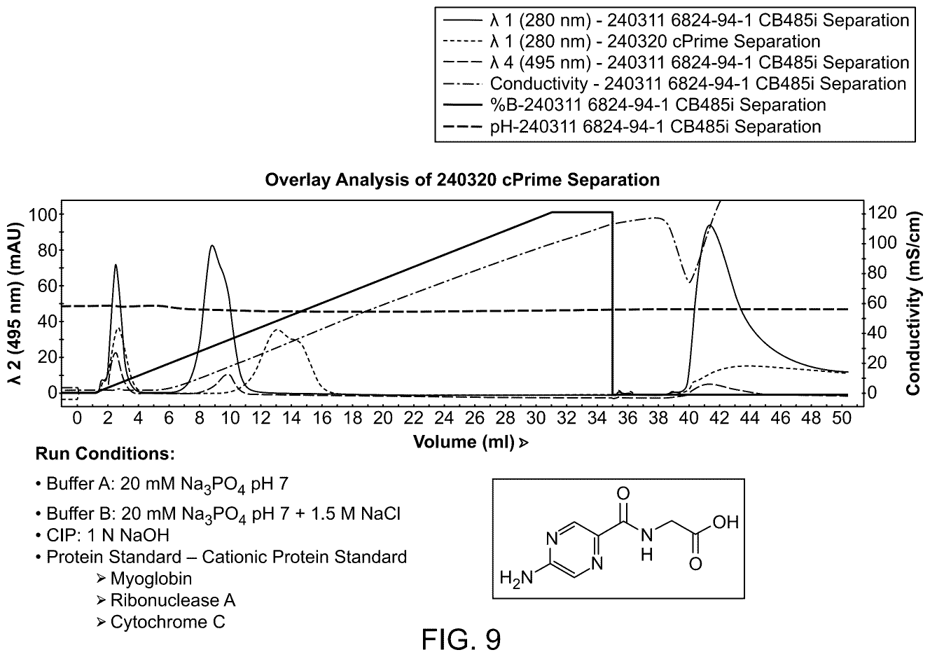

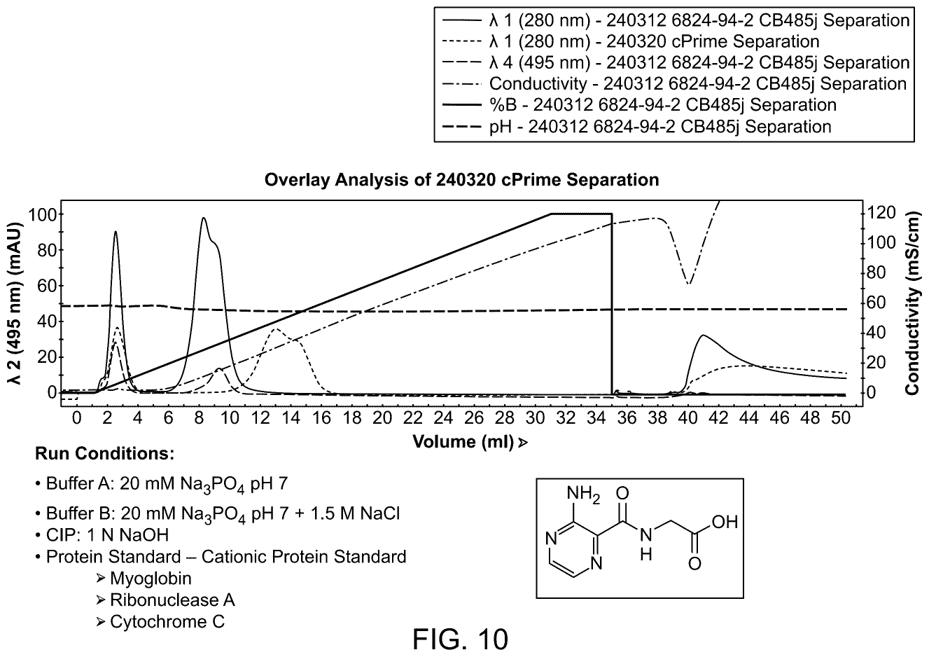

A 2.2 mL column was packed with various immobilized ligands. Columns were equilibrated with 20 mM Na₃PO₄ (pH=7; Buffer A). A cationic protein standard of myoglobin, ribonuclease A, and cytochrome c was loaded onto the column in Buffer A. Elution was performed with a salt gradient comprising Buffer A and Buffer B (20 mM Na₃PO₄+1.5 M NaCl (pH=7)). The elution profiles using salt gradients for the various ligands are illustrated in FIGS. 1-10. Each column was loaded with 100 μL of Bio-Rad's Cation Protein Standard in Buffer A. This protein standard elutes in the order of myoglobin, ribonuclease A, and cytochrome c.

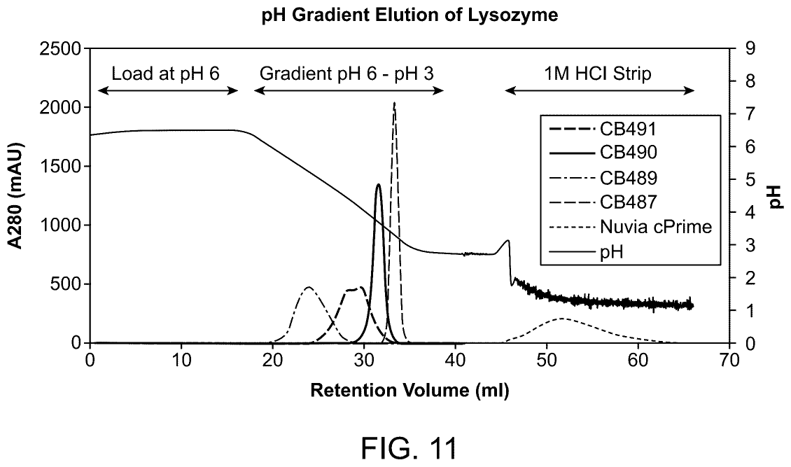

In FIG. 11, elution profiles for CB489, CB491, CB490, and CB487 compared to NcP using a pH gradient. Two (2) mL columns were equilibrated with 5 column volumes (CV) of Buffer B (50 mM citrate, pH 3.0) followed by 5 column volumes of buffer A (50 mM citrate, pH 6.0). A 250 μL sample of lysozyme (5 mg/ml lysozyme in Buffer A) was loaded onto the column after equilibration with Buffer A. A pH gradient of 0-100% Buffer B was applied to the column at a flow rate of about 2 mL/minute to elute bound protein (over about 10 CV). The pH gradient was not able to elute the protein on the NcP ligand until the column was stripped with 1M HCl (20 CV). CB489, CB491, CB490, CB482j, and CB487 were stripped with 5 CV of Buffer B.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

TABLE 1
Exemplary of Ligands and chromatography
(aminopyrazine carboxamido, aminopicolinamido, aminonicotinamido,
aminoisonicotinamido aminopyrimidine carboxamido derivatives)

| | | | Ionic |
|---|---|---|---|
| | | | Capacity |
| | | | (micro- |
| | Ligand | Structure of Chromatography | mole/ |
| Ligand Structure | Lot | Resin (spheres represent matrix) | mL) |
| 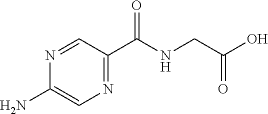 [SMILES: Nc1cnc(C(=O)NCC(=O)O)cn1] | CB485i | 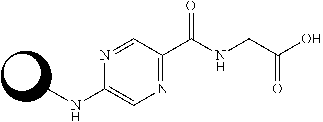 | 36 |
| (5-aminopyrazine-2-carbonyl)glycine | | | |
| 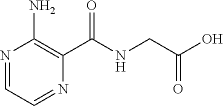 [SMILES: Nc1nccnc1C(=O)NCC(=O)O] (3-aminopyrazine-2-carbonyl)glycine | CB485j | 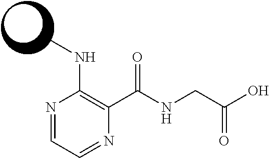 | 29 |
| 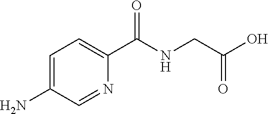 [SMILES: Nc1ccc(C(=O)NCC(=O)O)nc1] | CB482c | 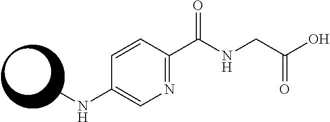 | 84 |
| (5-aminopicolinoyl)glycine | | | |
| 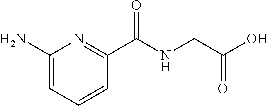 [SMILES: Nc1cccc(C(=O)NCC(=O)O)n1] | CB488 | 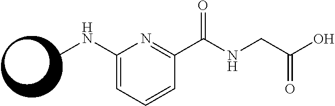 | 68 |
| (6-aminopicolinoyl)glycine | | | |
| 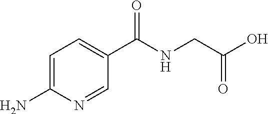 [SMILES: Nc1ccc(C(=O)NCC(=O)O)cn1] | CB482e | 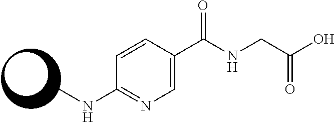 | 36 |
| (6-aminonicotinoyl)glycine | | | |
| 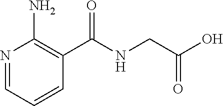 [SMILES: Nc1ncccc1C(=O)NCC(=O)O] (2-aminonicotinoyl)glycine | CB489 | 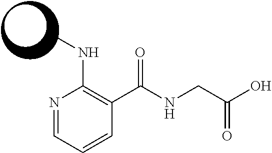 | 33 |
| 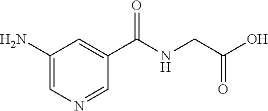 [SMILES: Nc1cncc(C(=O)NCC(=O)O)c1] | CB487 | 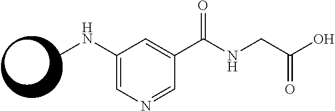 | 63 |
| (5-aminonicotinoyl)glycine | | | |
| 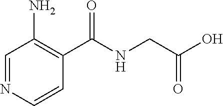 [SMILES: Nc1cnccc1C(=O)NCC(=O)O] (3-aminoisonicotinoyl)glycine | CB490 | 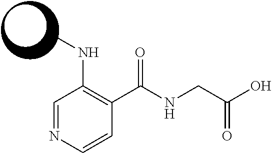 | 44 |
| 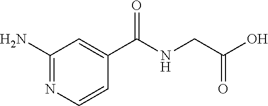 [SMILES: Nc1cc(C(=O)NCC(=O)O)ccn1] | CB491 | 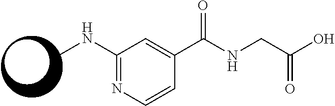 | 35 |
| (2-aminoisonicotinoyl)glycine | | | |
| 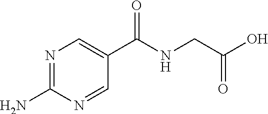 [SMILES: Nc1ncc(C(=O)NCC(=O)O)cn1] | | 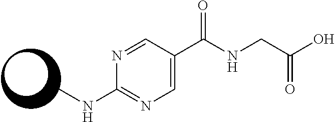 | |
| (2-aminopyrimidine-5-carbonyl)glycine | | | |
| 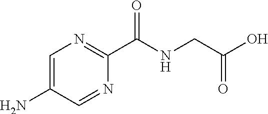 [SMILES: Nc1cnc(C(=O)NCC(=O)O)nc1] | | 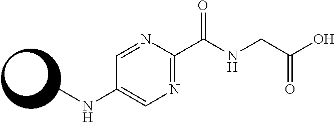 | |
| (5-aminopyrimidine-2-carbonyl)glycine | | | |
| 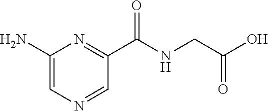 [SMILES: Nc1cncc(C(=O)NCC(=O)O)n1] | | 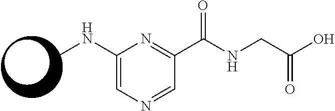 | |
| (6-aminopyrazine-2-carbonyl)glycine | | | |
| 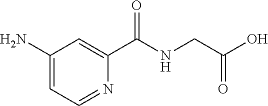 [SMILES: Nc1ccnc(C(=O)NCC(=O)O)c1] | | 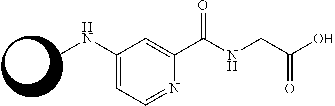 | |
| (4-aminopicolinoyl)glycine | | | |
| 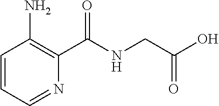 [SMILES: Nc1cccnc1C(=O)NCC(=O)O] (3-aminopicolinoyl)glycine | | 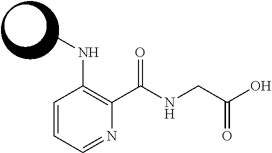 | |
| 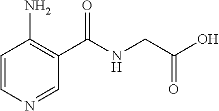 [SMILES: Nc1ccncc1C(=O)NCC(=O)O] (4-aminonicotinoyl)glycine | | 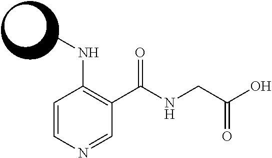 | |
| 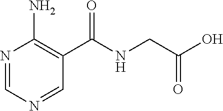 [SMILES: Nc1ncncc1C(=O)NCC(=O)O] (4-aminopyrimidine-5-carbonyl)glycine | | 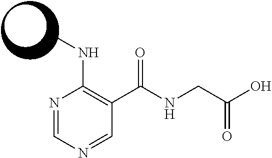 | |
| 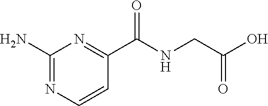 [SMILES: Nc1nccc(C(=O)NCC(=O)O)n1] | | 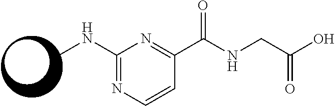 | |
| (2-aminopyrimidine-4-carbonyl)glycine | | | |
| 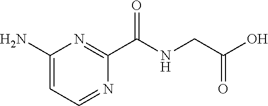 [SMILES: Nc1ccnc(C(=O)NCC(=O)O)n1] | | 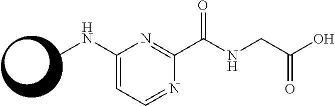 | |
| (4-aminopyrimidine-2-carbonyl)glycine | | | |
| 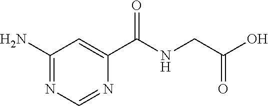 [SMILES: Nc1cc(C(=O)NCC(=O)O)ncn1] | | 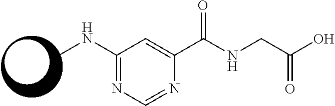 | |
| (6-aminopyrimidine-4-carbonyl)glycine | | | |
| 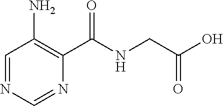 [SMILES: Nc1cncnc1C(=O)NCC(=O)O] (5-aminopyrimidine-4-carbonyl)glycine | | 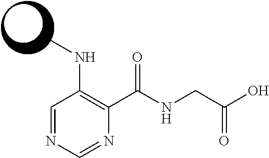 | |

We claim:

1. A ligand of the structure: 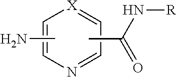
where X is CH or N; or 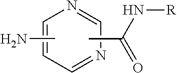
and for each ligand: 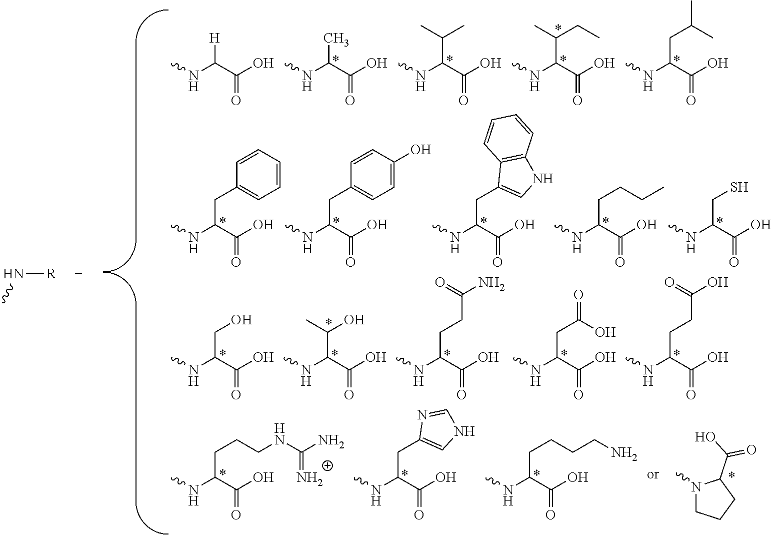
and the * represents a stereogenic carbon atom.

2. The ligand according to claim 1, wherein said ligand is:

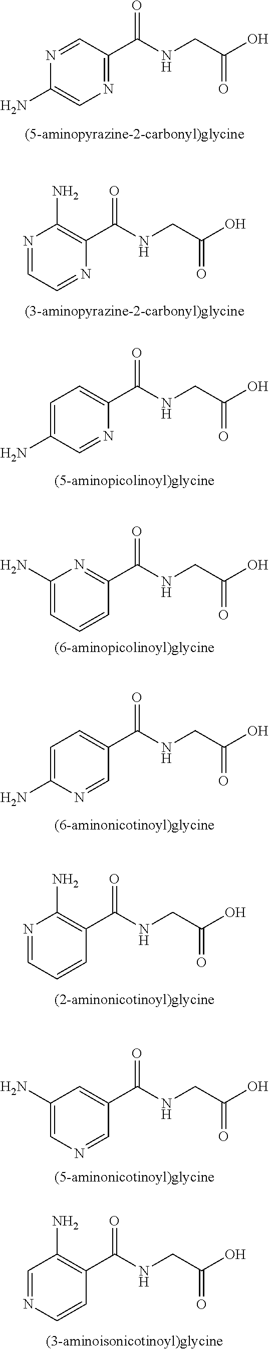

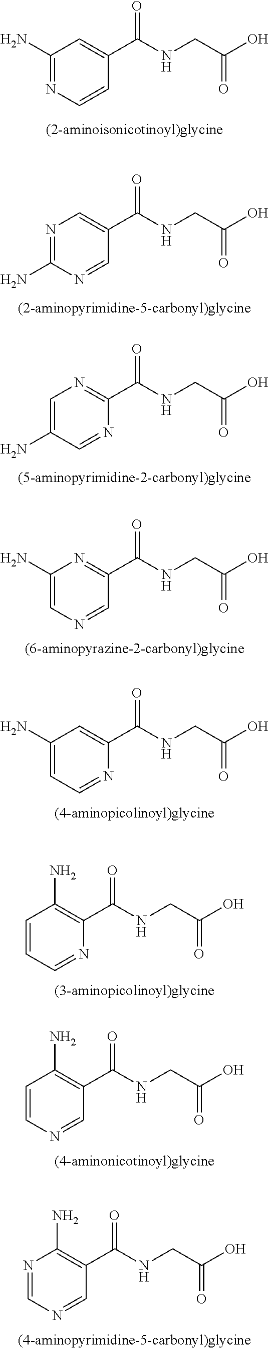

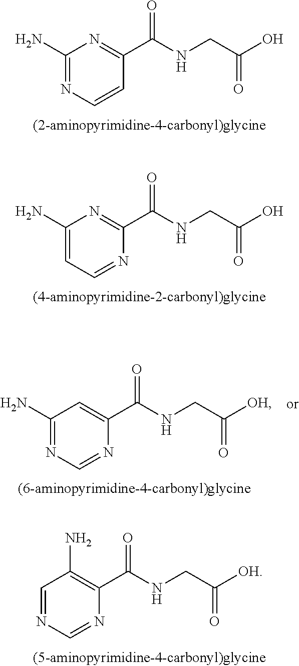 [SMILES: Nc1nccc(C(=O)NCC(=O)O)n1 ; Nc1ccnc(C(=O)NCC(=O)O)n1 ; Nc1cc(C(=O)NCC(=O)O)ncn1 ; Nc1cncnc1C(=O)NCC(=O)O]

3. The ligand according to claim 1, wherein the ligand is a chiral ligand and the stereogenic carbon of the group R that is alpha to the amide group has a D-configuration or has an L-configuration.

4. A mixed mode charge induction IEX chromatography (MMCIIC) chromatography resin comprising:
a support matrix, an optional linker and one or more ligand according to claim 1, the one or more ligand being coupled to the support matrix via the primary amine group of the ligand to form a mixed mode charge induction IEX chromatography (MMCIIC) resin.

5. The MMCIIC chromatography resin according to claim 4, wherein the one or more ligand is a chiral ligand and the stereogenic carbon of the group R that is alpha to the amide group has a D-configuration or has an L-configuration.

6. The MMCIIC chromatography resin according to claim 4, said one or more ligand being attached to the support matrix by a linker.

7. The MMCIIC chromatography resin according to claim 6, said one or more ligand being attached to the support matrix by an alkyl linker that is a straight or branched, saturated, aliphatic divalent radical having between 1-10 carbon atoms.

8. The MMCIIC chromatography resin according to claim 6, wherein the alkyl linker is a methyl, ethyl, propyl, isopropyl, butyl, isobutyl, sec-butyl, tert-butyl, pentyl, isopentyl, or hexyl divalent radical.

9. A mixed mode charge induction IEX chromatography (MMCIIC) chromatography medium comprising a solid support and a ligand and having the formula: 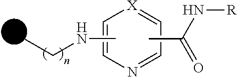
where n represents an alkyl group of 1 to 10 carbon atoms covalently bonded to the solid support (sphere) and the nitrogen atom, X is CH or N; or 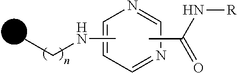
where n represents an alkyl group of 1 to 10 carbon atoms covalently bonded to the solid support (sphere) and the nitrogen atom; or 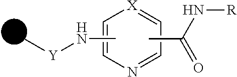
where Y represents an alkyl linker that is a straight or branched, saturated, aliphatic divalent radical having between 1-10 carbon atoms that is covalently bonded to the solid support (sphere) and nitrogen atom, X is CH or N; or 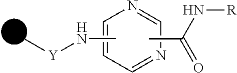
where Y represents an alkyl linker that is a straight or branched, saturated, aliphatic divalent radical having between 1-10 carbon atoms that is covalently bonded to the solid support (sphere) and nitrogen atom; and 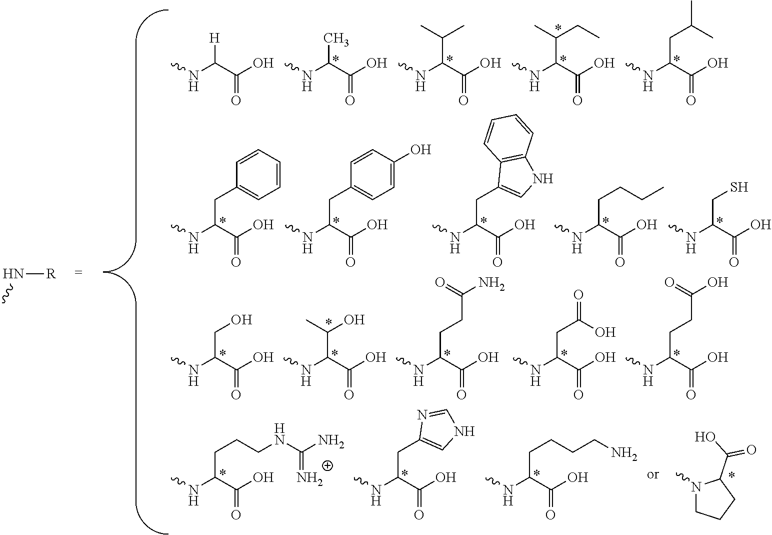
and the * representing a stereogenic carbon atom.

10. The mixed-mode chromatography medium according to claim 9, wherein the ligand is a chiral ligand and the stereogenic carbon of the group R that is alpha to the amide group has a D-configuration or has an L-configuration.

11. The mixed-mode chromatography medium according to claim 9, wherein the mixed mode chromatography medium is selected from the group consisting of: 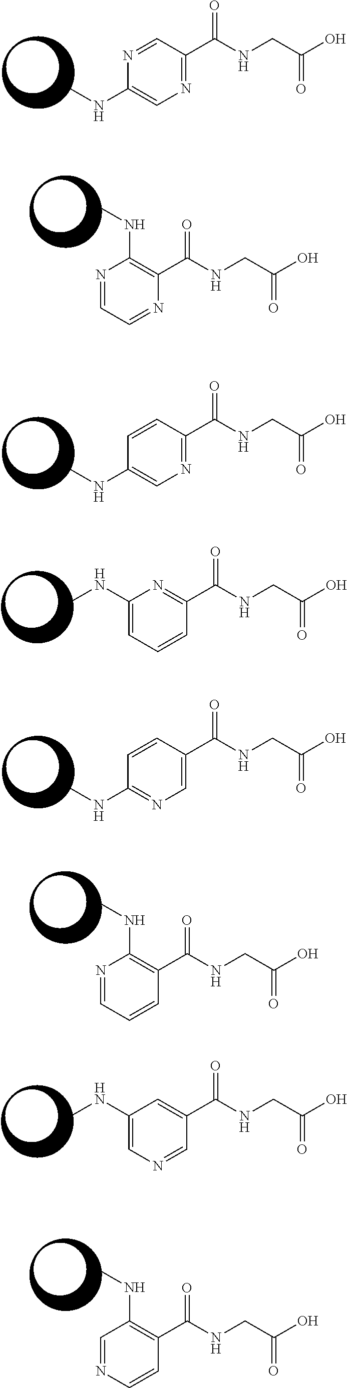 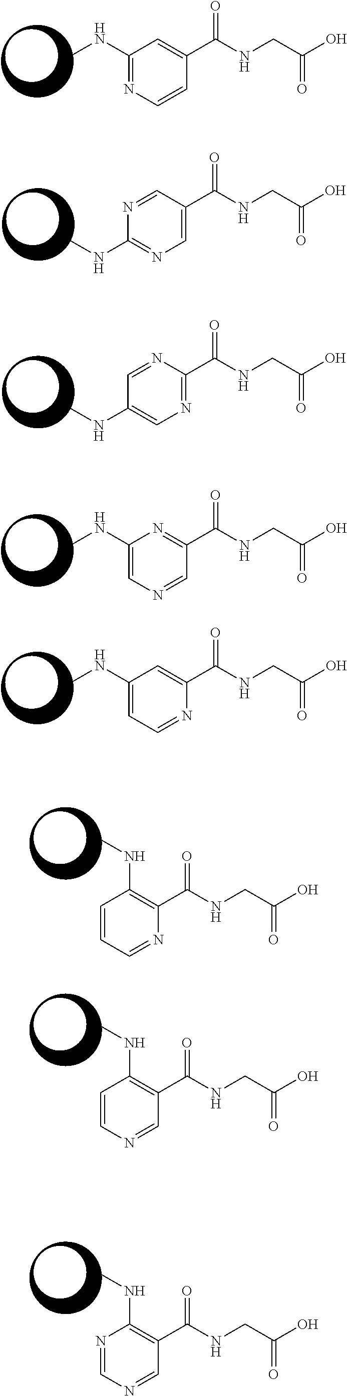 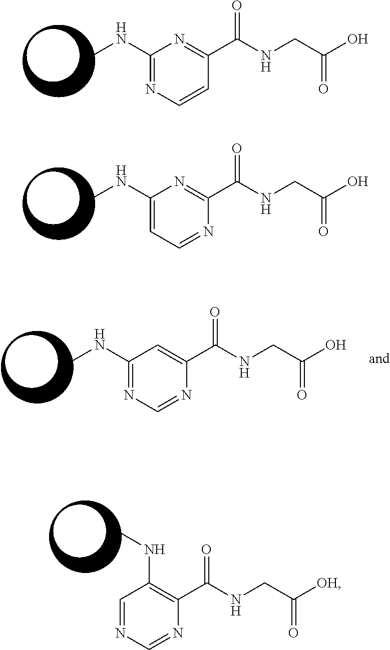
wherein the black bead and line represents the solid support and linker covalently bonded to the nitrogen atom.

12. A method for manufacturing a mixed-mode chromatography medium, said method comprising: (a) oxidizing diol groups on diol-functionalized solid support, thereby converting said diol-functionalized solid support to an aldehyde-functionalized solid support; and (b) coupling one or more amine-functionalized ligand according to claim 1 to said aldehyde-functionalized solid support.

13. The method according to claim 12, wherein a single amine-functionalized ligand is coupled to said aldehyde-functionalized solid support.

14. The method according to claim 12, wherein the ligand(s) is/are a chiral ligand and the stereogenic carbon of the group R that is alpha to the amide group has a D-configuration or has an L-configuration.

15. A method for purifying a protein from a source solution, said method comprising: (a) contacting said source solution with a mixed mode charge induction IEX chromatography (MMCIIC) mixed-mode chromatography medium according to claim 9 under conditions that permit said protein to bind to said MMCIIC medium; and (b) eluting said protein so bound from said MMCIIC medium or chromatography resin.

16. The method according to claim 15, wherein the protein is eluted using a pH gradient.

17. The method according to claim 15, wherein the protein is eluted using a salt gradient.

18. The method according to claim 15, wherein the chromatography medium comprises a ligand or ligands that are chiral and the stereogenic carbon of the group R that is alpha to the amide group has a D-configuration or has an L-configuration.

19. A method for purifying a protein from a source solution, said method comprising: (a) contacting said source solution with a mixed mode charge induction IEX chromatography (MMCIIC) mixed-mode chromatography medium according to claim 9 under conditions which cause the protein to not bind to said MMCIIC medium; and (b) collecting said protein without it having been bound to said solid support, separating it from molecules that are bound.

20. The method according to claim 19, wherein the chromatography medium comprises a ligand or ligands that are chiral and the stereogenic carbon of the group R that is alpha to the amide group has a D-configuration or has an L-configuration.
